(19) United States
(12) Patent Application Publication
(10) Pub. No.: US 2023/0270988 A1
(43) Pub. Date: Aug. 31, 2023

(54) DUAL ARM HEMOSTASIS VALVE HUB FOR INTRODUCER SHEATHS

(71) Applicants: Boston Scientific Medical Device Limited, Galway (IE); Boston Scientific Scimed Inc., Maple Grove, MN (US)

(72) Inventors: Neeraj Kumar Sharma, New Delhi (IN); Sumit Agrawal, Haryana (IN); Rupesh Kumar Pahwa, Gurugram (IN); Qian Liu, Plymouth, MN (US)

(21) Appl. No.: 18/114,025

(22) Filed: Feb. 24, 2023

Related U.S. Application Data
(60) Provisional application No. 63/314,178, filed on Feb. 25, 2022.

(51) Int. Cl. A61M 39/06; A61M 60/13; A61M 60/216; A61M 60/865

(52) U.S. Cl. CPC A61M 39/06; A61M 60/13; A61M 60/216; A61M 60/865; A61M 2039/0258

(57) ABSTRACT

A hemostasis valve hub for a sheath, such as an introducer sheath, includes a hub base including a first arm defining a first lumen and having a distal end and a proximal end and a second arm extending from the first arm and defining a second lumen and having a distal end and a proximal end, a seal positioned within the first lumen and adjacent a lock nut, the lock nut engaged with the proximal end of the first arm and positioned at least partially within the first lumen, a primary seal positioned adjacent the proximal end of the first arm, and a secondary seal positioned adjacent the proximal end of the second arm.

### CROSS-REFERENCE TO RELATED APPLICATION

This application claims priority to Provisional Application No. 63/314,178, filed Feb. 25, 2022, which is herein incorporated by reference in its entirety.

### TECHNICAL FIELD

The present disclosure relates to a hub for an introducer sheath. More specifically, the present disclosure relates to a dual arm hemostasis valve hub for large bore introducer sheaths.

### BACKGROUND

In various procedures for delivering intravascular medical devices, an introducer sheath is inserted into a blood vessel of a patient, for example a femoral artery, and medical devices are inserted into the introducer sheath for introduction into the blood vessel. In various instances, the medical devices include catheters or other medical devices such as a blood pump. In various instances, multiple medical devices need to be introduced into or inserted through the blood vessel at the same time. A hemostasis valve hub may be incorporated at a proximal end of the large bore introducer sheath to reduce blood leakage as devices are being inserted, positioned, and removed. There is a need for an optimized hemostasis valve hub that allows for the passage of multiple devices into the introducer sheath into a single blood vessel access site, while minimizing blood leakage and reducing axial displacement of the medical devices within the valve hub while facilitating passage of the devices through an introducer sheath.

### SUMMARY

In an Example 1, a hemostasis valve hub for a sheath includes a hub base including a first arm defining a first lumen and having a distal end and a proximal end and a second arm extending from the first arm and defining a second lumen and having a distal end and a proximal end, a seal positioned within the first lumen and adjacent a lock nut, the lock nut engaged with the proximal end of the first arm and positioned at least partially within the first lumen, a primary seal positioned adjacent the proximal end of the first arm, and a secondary seal positioned adjacent the proximal end of the second arm.

In an Example 2, the hemostasis valve hub of Example 1 further includes wherein the hemostasis valve hub further includes a securing mechanism engaged with the hub base to secure the hemostasis valve hub to the sheath.

In an Example 3, the hemostasis valve hub of Example 2 further includes wherein the securing mechanism is a ferrule positioned within the hub base.

In an Example 4, the hemostasis valve hub of Example 2 further includes wherein the hub base includes a narrowed portion and the securing mechanism includes a threaded cap engaged with the narrowed portion and the sheath.

In an Example 5, the hemostasis valve hub any one of the preceding Examples further includes wherein the lock nut is configured to threadedly engage with the first arm and further includes an inner portion configured to engage with the seal.

In an Example 6, the hemostasis valve hub of Example 5 further includes wherein the inner portion of the lock nut is configured to axially compress the seal to decrease a size of a lumen the seal.

In an Example 7, the hemostasis valve hub of any one of the of the preceding Examples further includes wherein the hub further includes a primary cap engaged with the lock nut and a secondary cap engaged with the proximal end of the second arm.

In an Example 8, the hemostasis valve hub of any one of the preceding Examples further includes wherein the primary seal has a diameter and the secondary seal has a diameter, the diameter of the primary seal being greater than the diameter of the secondary seal.

In an Example 9, a delivery system for a plurality of medical devices into a blood vessel includes a sheath for insertion into the blood vessel, the sheath having a proximal end and a distal end and a hemostasis valve hub for attachment to the proximal end of the sheath. The hemostasis valve hub includes a hub base including a first arm defining a first lumen and having a distal end and a proximal end and a second arm extending from the first arm and defining a second lumen and having a distal end and a proximal end, a seal positioned within the first lumen and adjacent a lock nut, the lock nut engaged with the proximal end of the first arm and positioned at least partially within the first lumen, a primary seal positioned adjacent the proximal end of the first arm and a primary cap engaged with the lock nut, and a secondary seal positioned adjacent the proximal end of the second arm.

In an Example 10, the hemostasis valve hub of Example 9 further includes wherein the primary seal has a first diameter, the secondary seal has a second diameter, and the first diameter is greater than the second diameter.

In an Example 11, the hemostasis valve hub of Example 9 or Example 10 further includes wherein the hub base includes a securing mechanism for securing to the proximal end of the sheath.

In an Example 12, the hemostasis valve hub of Example 11 further includes wherein the securing mechanism is a ferrule positioned within the hub based of the hemostasis valve hub.

In an Example 13, a method of assembly for a sheath and a hub includes placing a hub base onto a proximal end of a sheath, the hub base having a first arm and a second arm, the first arm defining a first lumen and a second arm defining a second lumen, attaching a securing mechanism to the hub base and the proximal end of the sheath, and inserting a seal into the first lumen of the first arm. The method further includes engaging a lock nut, a primary seal and a primary cap with the first arm and engaging a secondary seal with the second arm.

In an Example 14, the method of Example 13 further includes wherein the seal includes a lumen configured to receive a medical device.

In an Example 15, the method of Example 13 or Example 14 further includes wherein the securing mechanism is a ferrule positioned within the hub base.

In an Example 16, a hemostasis valve hub for a sheath includes a hub base including a first arm defining a first lumen and having a distal end and a proximal end and a second arm extending from the first arm and defining a second lumen and having a distal end and a proximal end, a seal positioned within the first lumen and adjacent a lock nut, the lock nut engaged with the proximal end of the first arm and positioned at least partially within the first lumen, a primary seal positioned adjacent the proximal end of the first arm and a primary cap engaged with the lock nut, and a secondary seal positioned adjacent the proximal end of the second arm.

In an Example 17, the hemostasis valve hub of Example 16 further includes wherein the hemostasis valve hub further includes a securing mechanism engaged with the hub base to secure the hemostasis valve hub to the sheath.

In an Example 18, the hemostasis valve hub of Example 17 wherein the securing mechanism is a ferrule positioned within the hub base.

In an Example 19, the hemostasis valve hub of Example 17 further includes wherein the hub base includes a narrowed portion and the securing mechanism includes a threaded cap engaged with the narrowed portion and the sheath.

In an Example 20, the hemostasis valve hub of Example 16 further includes wherein the lock nut includes an inner portion configured to engage with the seal.

In an Example 21, the hemostasis valve hub of Example 20 further includes wherein the inner portion of the lock nut is configured to axially compress the seal to decrease the size of a lumen of the seal.

In an Example 22, the hemostasis valve hub of Example 16 further includes wherein the lock nut is configured to threadedly engage with the first arm.

In an Example 23, the hemostasis valve hub of Example 16 further includes wherein the primary seal has a diameter and the secondary seal has a diameter, the diameter of the primary seal being greater than the diameter of the secondary seal.

In an Example 24, the hemostasis valve hub of Example 16 further includes wherein the primary seal has a thickness and the secondary seal has a thickness, the thickness of the primary seal being greater than the thickness of the secondary seal.

In an Example 25, a delivery system for a plurality of medical devices into a blood vessel includes a sheath for insertion into the blood vessel, the sheath having a proximal end and a distal end and a hemostasis valve hub for attachment to the proximal end of the sheath. The hemostasis valve hub includes a hub base including a first arm defining a first lumen and having a distal end and a proximal end and a second arm extending from the first arm and defining a second lumen and having a distal end and a proximal end, a seal positioned within the first lumen and adjacent a lock nut, the lock nut engaged with the proximal end of the first arm and positioned at least partially within the first lumen, a primary seal positioned adjacent the proximal end of the first arm and a primary cap engaged with the lock nut, and a secondary seal positioned adjacent the proximal end of the second arm.

In an Example 26, the delivery system of Example 25 further includes wherein the primary seal has a first diameter, the secondary seal has a second diameter, and the first diameter is greater than the second diameter.

In an Example 27, the delivery system of Example 25 further includes wherein the hub base includes a securing mechanism for securing to the proximal end of the sheath.

In an Example 28, the delivery system of Example 27 further includes wherein the securing mechanism is a ferrule positioned within the hub based of the hemostasis valve hub.

In an Example 29, the delivery system of Example 27 further includes wherein the securing mechanism is composed of a narrowed portion of the hub base having a threaded portion and a threaded cap configured for attachment to the threaded portion.

In an Example 30, the delivery system of Example 25 further includes wherein the engagement of the lock nut and the seal causes a lumen of the seal to decrease in size.

In an Example 31, a method of assembly for a sheath and a hub includes placing a hub base onto a proximal end of a sheath, the hub base having a first arm and a second arm, the first arm defining a first lumen and a second arm defining a second lumen, attaching a securing mechanism to the hub base and the proximal end of the sheath, and inserting a seal into the first lumen of the first arm. The method further includes engaging a lock nut, a primary seal and a primary cap with the first arm and engaging a secondary seal with the second arm.

In an Example 32, the method of Example 31 further includes wherein the seal includes a lumen configured to receive a medical device.

In an Example 33, the method of Example 31 further includes wherein the securing mechanism is a ferrule positioned within the hub base.

In an Example 34, the method of Example 31 further includes wherein the securing mechanism includes a threaded cap configured for engaging with the proximal end of the sheath and a narrowed portion of the hub base.

In an Example 35, the method of Example 31 further includes wherein the lock nut includes an inner portion configured to engage with the seal and axially compress the seal to decrease the size of the lumen of the seal.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a cross sectional view of an introducer sheath after insertion into a blood vessel, in accordance with embodiments of the present disclosure.

FIG. 2 illustrates a cross sectional view of the introducer sheath after insertion into a blood vessel and insertion of a medical device into the introducer sheath, in accordance with embodiments of the present disclosure.

FIG. 3 illustrates a side view of a hemostasis valve hub for use with an introducer sheath, in accordance with embodiments of the present disclosure.

FIG. 4 illustrates a cross sectional view of the hemostasis valve hub as shown in FIG. 3, in accordance with embodiments of the present disclosure.

FIG. 5 illustrates an exploded view of the hemostasis valve as shown in FIGS. 3-4, in accordance with embodiments of the present disclosure.

FIG. 6 illustrates an exploded view of a hemostasis valve hub for use with an introducer sheath, in accordance with embodiments of the present disclosure.

FIG. 7 illustrates an enlarged view of a main cap for use with a hemostasis valve hub, in accordance with embodiments of the present disclosure.

FIG. 8 illustrates an enlarged view of a lock nut for use with a hemostasis valve hub, in accordance with embodiments of the present disclosure.

FIG. 9 illustrates an enlarged view of a seal for use with a hemostasis valve hub, in accordance with embodiments of the present disclosure.

FIG. 10A illustrates an enlarged view of an additional seal for use with a hemostasis valve hub, in accordance with embodiments of the present disclosure.

FIG. 10B illustrates a cross sectional view of the seal of FIG. 10A, in accordance with embodiments of the present disclosure.

FIG. 10C illustrates an additional cross-sectional view of the seal of FIG. 10A, in accordance with embodiments of the present disclosure.

FIG. 10D illustrates an enlarged view of an additional seal for use with a hemostasis valve hub, in accordance with embodiments of the present disclosure.

FIG. 10E illustrates a cross sectional view of the seal of FIG. 10D, in accordance with embodiments of the present disclosure.

FIG. 10F illustrates an additional cross-sectional view of the seal of FIG. 10D, in accordance with embodiments of the present disclosure.

FIG. 11 illustrates a flow chart of a method of assembly of an introducer sheath and a hub, in accordance with embodiments of the present disclosure.

### DETAILED DESCRIPTION

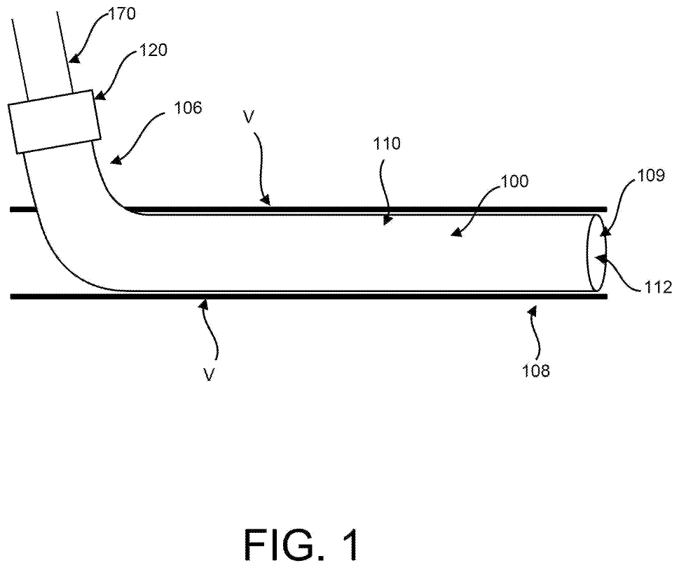

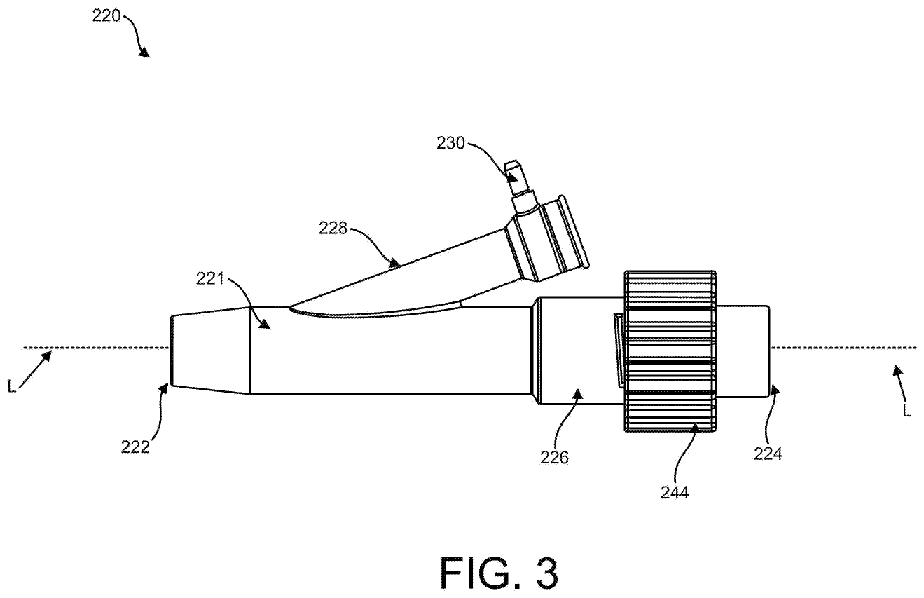

FIG. 1 illustrates a side cross sectional view of a blood vessel V with a sheath 100, such as an introducer sheath 100, inserted at least partially into the blood vessel V. In some embodiments, the introducer sheath 100 is used for facilitating the passage of various relatively large medical devices, such as a blood pump, as will be described further herein, through the introducer sheath 100 and into the blood vessel V. Hence, the introducer sheath 100 may be referred to as a large bore introducer sheath. Specifically, the introducer sheath 100 comprises a proximal end 106 and a distal end 108 that is opposite the proximal end 106. The introducer sheath 100 includes a proximal opening (not shown) adjacent the proximal end 106 and a distal opening 109 adjacent the distal end 108. A body portion 110 of the introducer sheath 100 extends between the proximal end 106 and the distal end 108, and the body portion 110 defines a lumen 112 of the introducer sheath 100. The introducer sheath 100 may be formed by various polymeric or metallic materials. In further embodiments, the introducer sheath 100 may comprise an additional surface coating. The surface coating may include, but is not limited to, silicone, PET, or any other applicable polymer. A hub 120 is commonly included at the proximal opening. The hub 120, also referred to herein as a hemostasis valve hub, is configured for hemostasis, i.e, to prevent blood from leaking out of the introducer sheath 100 during use. In various instances, it may be desired for the passage of multiple medical devices through the introducer sheath 100 at one time. As such, in some embodiments as will be disclosed further with reference to FIGS. 3-8, the hemostasis valve hub 120 may include at least two arms having openings for allowing the insertion of at least two devices into the introducer sheath 100 at once, without requiring removal of the first device and subsequent insertion of the second device. The dual arm configuration allows the insertion of the two devices while also maintaining hemostasis and reducing the blood leakage from the introducer sheath 100 and/or the hub 120 during the insertion, use of, or removal of the various medical devices. In some embodiments, two catheters are inserted into the hub 120, and various other medical devices may be inserted into at least one of the catheters for delivery into the blood vessel V. As shown in FIG. 1, the hub 120 may be used to receive a catheter 170. The catheter 170 extends through the hub 120 and the introducer sheath 100 and may couple to a proximal end of a blood pump which may be inserted into the introducer sheath 100, as will be described further herein.

In some embodiments, the sheath 100 may be a repositioning sheath.

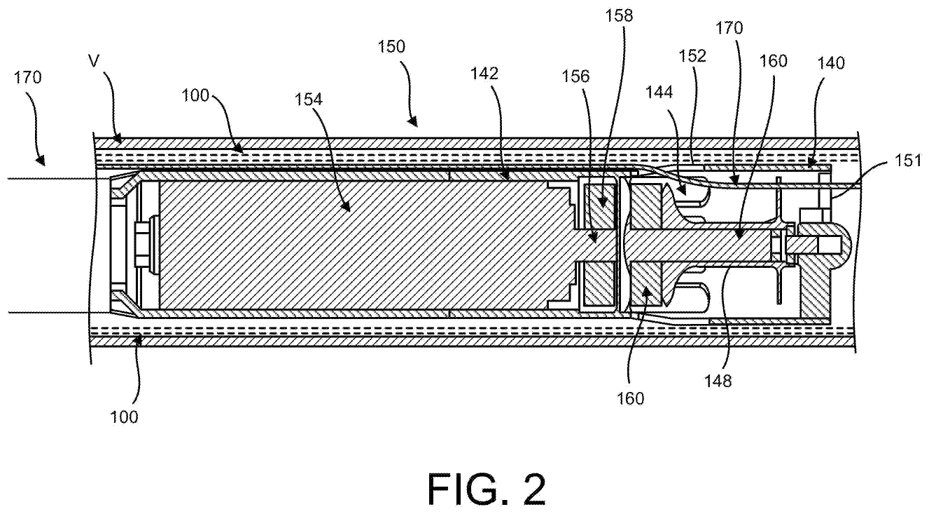

FIG. 2 illustrates a cross-sectional view of the introducer sheath 100 of FIG. 1 after insertion of a medical device, illustratively a blood pump 150, into the introducer sheath 100. The blood pump 150 generally includes an impeller assembly housing 140 and a motor housing 142. In some embodiments, the impeller assembly housing 140 and the motor housing 142 may be integrally or monolithically constructed. The impeller assembly housing 140 carries an impeller assembly 144 therein. The impeller assembly 144 includes an impeller shaft 146 and an impeller 148 that rotates relative to the impeller assembly housing 140 to drive blood through the blood pump 150. More specifically, the impeller 148 causes blood to flow from a blood inlet 151 formed on the impeller assembly housing 140, through the impeller assembly housing 140, and out of a blood outlet 152 formed on the impeller assembly housing 140. In some embodiments the impeller shaft 146 and the impeller 148 may be integrated, and in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may couple to a distally extending cannula (not shown), and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly housing 140. More specifically, in embodiments incorporating the impeller shaft 146, the impeller shaft 146 and the impeller 148 are configured to rotate with the driven magnet 160. In other embodiments, the motor 154 may couple to the impeller assembly housing 140 via other components. Additionally, as illustrated in FIG. 2, the catheter 170 extends from a proximal end of the blood pump 150. In some embodiments, the catheter 170 may be coupled to the motor housing 142 through a tapering connector and/or various other connecting means. The catheter 170 may have a flexible construction to facilitate to the delivery of the blood pump 150. While the introducer sheath 100 is illustrated above with the use of the blood pump 150, various other medical devices may be used in conjunction with the introducer sheath 100 and the hemostasis valve hub 120. For example, a variation of a blood pump may be used in conjunction with the introducer sheath 100. In other examples, a device other than a blood pump may be incorporated.

With reference now to FIGS. 3-5, an embodiment of a hemostasis valve hub will be described in further detail. Specifically, FIGS. 3-5 illustrate a hemostasis valve hub 220 having a distal end 222 and a proximal end 224 and a longitudinal axis L extending between the distal end 222 and the proximal end 224. Additionally, the hemostasis valve hub 220 has a hub base 221 comprising a first arm 226 and a second arm 228 that extends outwardly from the first arm 226 at an angle relative to the longitudinal axis L. The angle relative to the longitudinal axis L may be greater than zero. Further, as illustrated, the second arm 228 also comprises an access port 230 extending from the second arm 228. The access port 230 may be used for insertion of fluids or tools configured for irrigation.

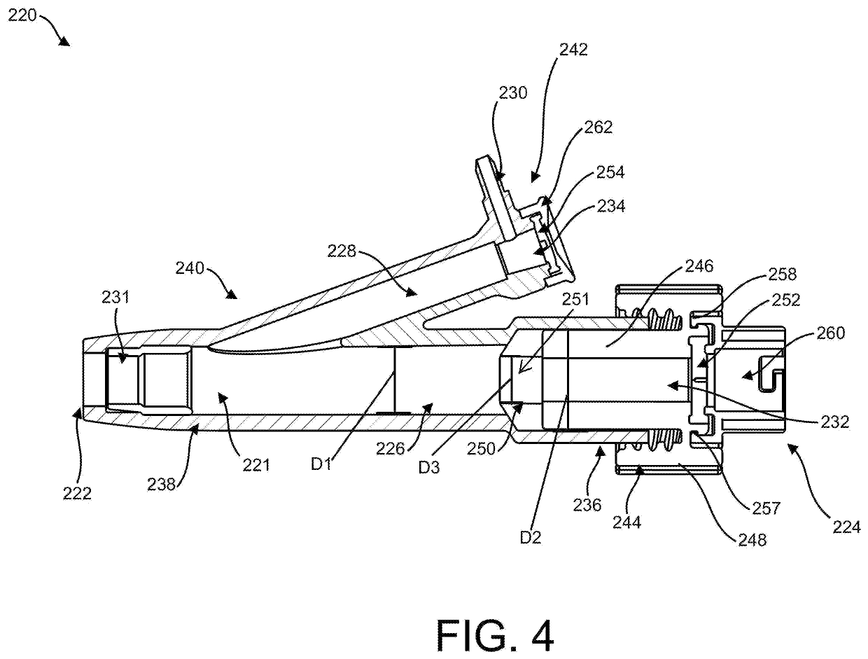

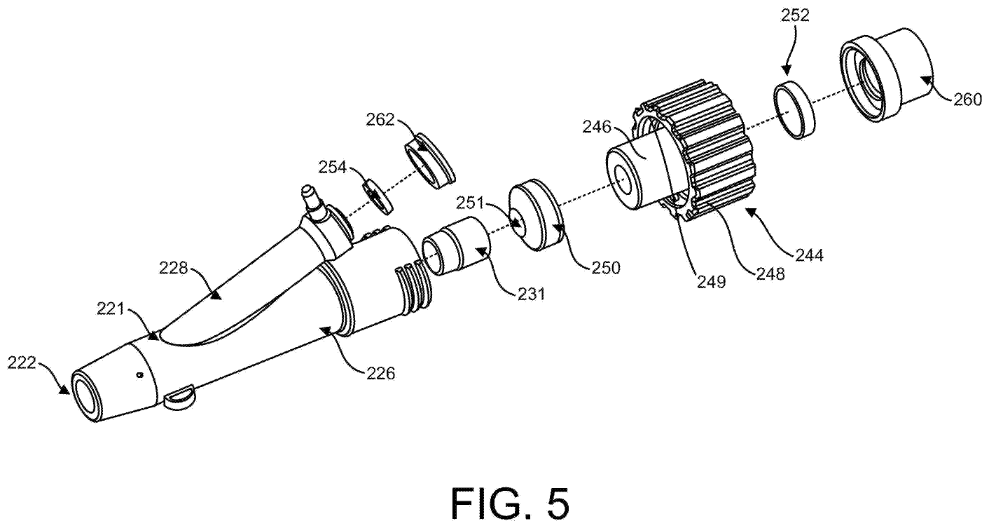

The distal end 222 of the hemostasis valve hub 220 is configured for attachment to the proximal end 106 (FIG. 1) of the introducer sheath 100 (FIG. 1), and more specifically with the proximal opening of the introducer sheath 100. As best illustrated in FIG. 4, the distal end 222 of the hemostasis valve hub 220 comprises a securing mechanism for securing the introducer sheath 100 within the distal end 222 of the hemostasis valve hub 220. For example, in the illustrative embodiment of FIGS. 4-5, the hemostasis valve hub 220 comprises a ferrule 231 positioned within the distal end 222 that is configured for securing the proximal flared opening of the introducer sheath 100 within hemostasis valve hub 220. The proximal end 224 of the hemostasis valve hub 220 is configured for receiving a plurality of medical devices for insertion through the hemostasis valve hub 220 and the introducer sheath 100 (FIG. 1). In other words, the configuration of the proximal end 224, and more specifically the configuration of the first arm 226 and the second arm 228, allows for at least two medical devices to be inserted into one access port of the patient simultaneously. In various embodiments, as previously described, the two medical devices may be two catheters or other medical devices, such as the blood pump 150 as previously disclosed with reference to FIG. 2, for insertion into the blood vessel V. The configuration of the hemostasis valve hub 220 allows for reduction of blood loss or access site inflammation that may otherwise occur if the first device needs to be removed and the second device subsequently removed. The hemostasis valve hub 220 also reduces the need for additional access ports to insert multiple medical devices into the blood vessel V.

The dual arm configuration of the hemostasis valve hub 220 will be described further herein. The first arm 226 comprises a first lumen 232 defined within the first arm 226 and extending generally parallel to the longitudinal axis L (FIG. 3). The second arm 228 comprises a second lumen 234 that extends through the second arm 228 and is generally angled to the longitudinal axis L. The first lumen 232 extends from a proximal end 236 of the first arm 226 which may correspond with the proximal end 224 of the hemostasis valve hub 220, to a distal end 238 of the first arm 226 which may correspond with the distal end 222 of the hemostasis valve hub 220. Further, the second lumen 234 extends between a distal end 240 of the second arm 228 and a proximal end 242 of the second arm 228.

With reference to FIGS. 4-5, the first arm 226 will now be described in further detail. As illustrated, the first lumen 232 comprises a first diameter D1 that extends from the distal end 238 and approaches the proximal end 236 of the first lumen 232. Toward the proximal end 236 of the first lumen 232, the first lumen 232 comprises a second diameter D2 that extends to the proximal end 236, wherein the diameter D2 is greater than the first diameter D1. As illustrated, the first lumen 232 comprises a seal 250 positioned adjacent a transition point of the first lumen 232 wherein the diameter increases from the first diameter D1 to the second diameter D2. The seal 250 defines a seal lumen 251 that extends through the seal 250 for receiving at least one medical device, as will be described further herein. Adjacent the seal 250 is a lock nut 244 that has an inner portion 246 extending at least partially into the first lumen 232 and capable of engagement with the seal 250. Lock nut 244 additionally comprises an outer portion 248 that is positioned at least partially around an outer surface of the first arm 226. More specifically, an inner surface 249 of the outer portion 248 of lock nut 244 may be threaded, and at least a portion of the outer surface of the first arm 226 may be threaded such that the lock nut 244 is threadedly engaged with the proximal end 236 of the first arm 226 to adjust and/or maintain the desired positioning of lock nut 244. In other words, the operator may rotate or screw the lock nut 244 into engagement with the hemostasis valve hub 220, while also causing engagement with the seal 250. As will be described further with reference to FIG. 9, the engagement of lock nut 244 with seal 250 causes inner portion 246 to engage with seal 250 and apply axial compression forces on the seal 250. As the seal 250 is axially compressed, the initial diameter D3′ (FIG. 9) of the lumen 251 decreases such that the lumen 251 decreases in size and is defined by a compressed diameter D3. As the diameter of the lumen 251 decreases in size, the seal 250 contacts and is tightened against a medical device (e.g., a catheter) passing through the seal 250 to stabilize the axial positioning of the medical device. Additionally, when the lock nut 244 axially compresses the seal 250, the seal 250 expands radially inwardly to provide a partial or full fluid seal against the exterior surface of the catheter, as well as radially outwardly to create a partial or full fluid seal against the inner surface of the hub base 221. In addition, the seal 250 may create various other radial and/or axial seals within the hub 220 as a result of axial compression and force against the seal 250.

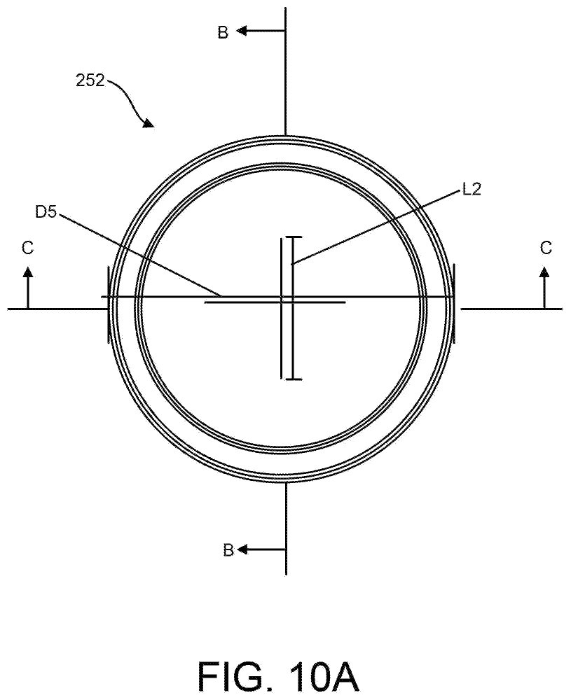

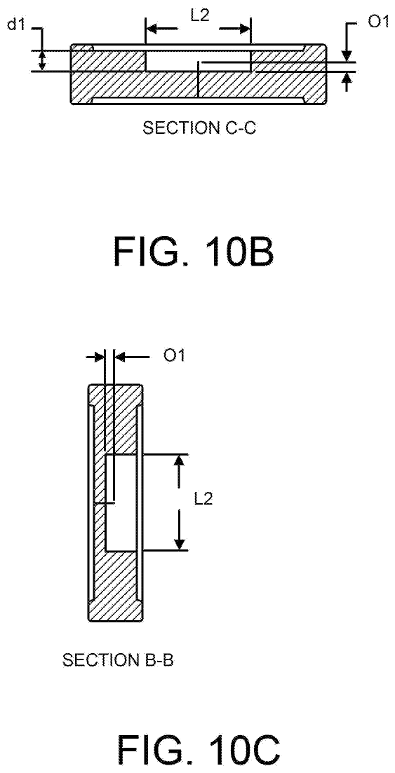

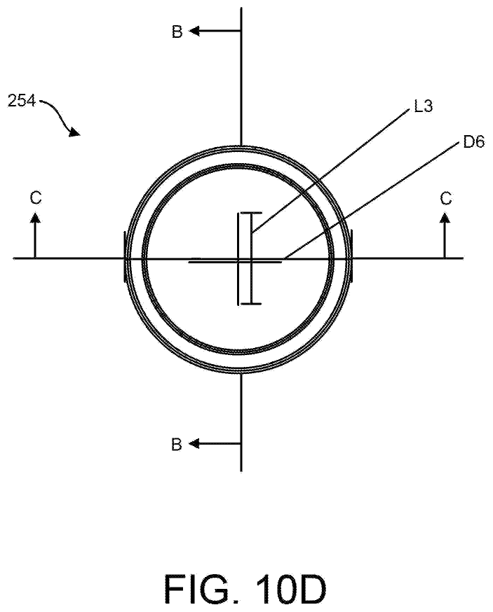

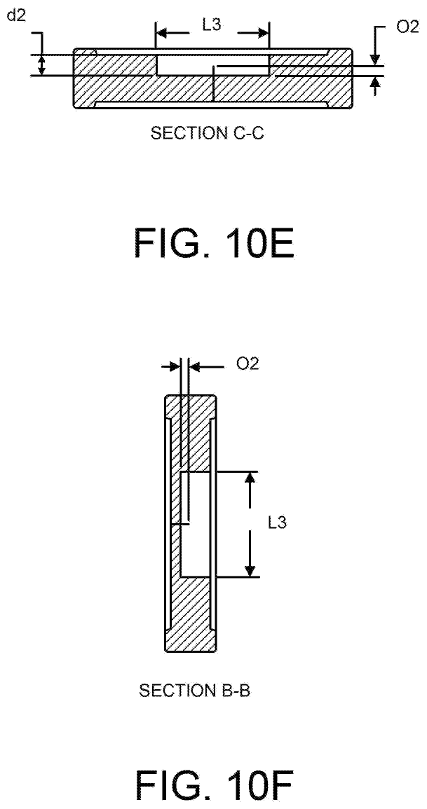

With continued reference to FIGS. 3-5, adjacent the lock nut 244 and positioned spaced proximally from the proximal end 236 from the first arm 226 is a primary seal 252. The primary seal 252 is configured such that it has a diameter that is less than a diameter of the lock nut 244, but greater than the first diameter D1 of the first lumen 232. As will be described further with reference to FIG. 10A, the primary seal 252 is configured for providing a fluid tight seal around the medical device passing through the hemostasis valve hub 220. The primary seal 252 is at least partially engaged with the lock nut 244 as shown in FIG. 4. Further, a primary cap 260 is positioned adjacent the primary seal 252 and engaged with the lock nut 244 such that the primary seal 252 is sandwiched between the lock nut 244 and the primary cap 260. More specifically, the lock nut 244 may have detents 257 configured for receiving protrusions 258 of the primary cap 260. This coupling ensures that the primary seal 252 is securely engaged to maintain a hemostatic seal after the insertion of medical devices into the hemostasis valve hub 220 and the introducer sheath 100 (FIG. 1). In some embodiments, the primary cap 260 may be configured to couple to a tightening port (not shown) used to secure axial movement of a medical device, such as a catheter, passing through the hub 220.

The second arm 228 will now be described further with continued reference to FIGS. 4-5. As previously described, the second arm 228 comprises a proximal end 242 and a distal end 240. The distal end 240 is configured for engaging with the first arm 226 such that the first lumen 232 and the second lumen 234 merge and become a singular lumen when passing through the distal end 222 of the hemostasis valve hub 220 and into the introducer sheath 100 (FIG. 1). The proximal end 242 of the second arm 228 is configured for maintaining a hemostatic seal upon insertion of an additional medical device into the second lumen 234. Specifically, adjacent the proximal end 242 of the second arm 228, the second arm 228 comprises a secondary seal 254 configured to span across an entire diameter of the second lumen 234. Additionally, positioned adjacent the secondary seal 254 and engaged with the second arm 228 is a secondary cap 262. The secondary cap 262 is configured to maintain the positioning of the secondary seal 254 against the second arm 228 of the hemostasis valve hub 220.

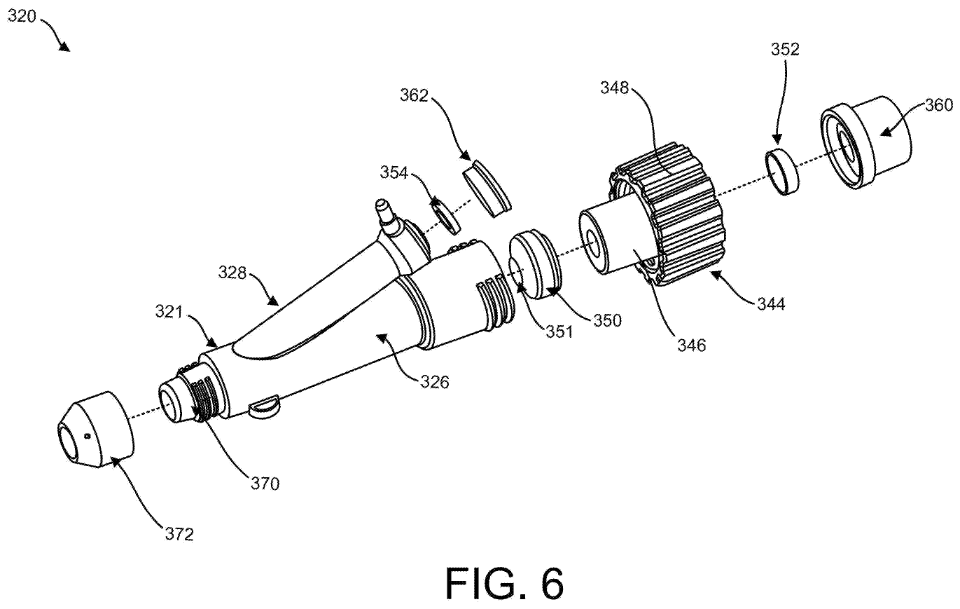

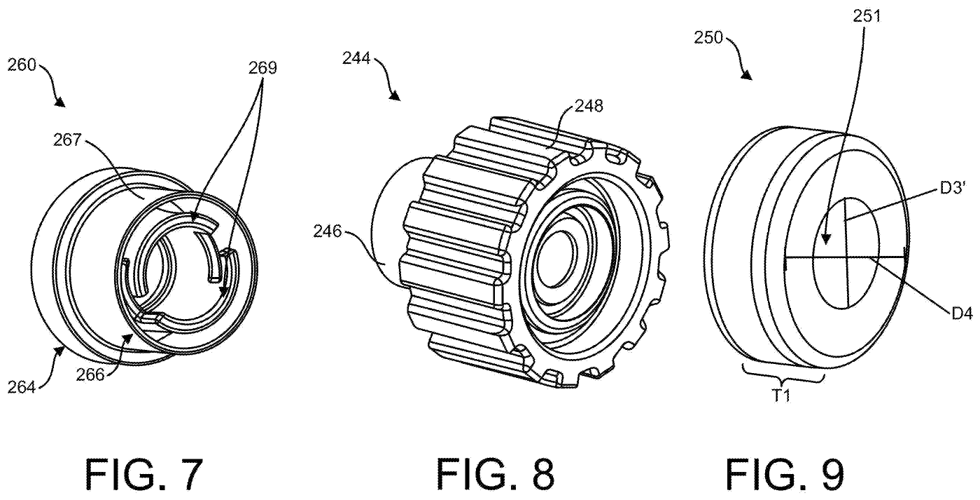

FIG. 6 illustrates an exploded view of an alternate embodiment of a hemostasis valve hub, illustratively a hemostasis valve hub 320. The hemostasis valve hub 320 may be similar to or the same as the hemostasis valve hub 220 described with reference to FIGS. 3-5, with the exception of the introducer sheath 100 securing mechanism at the distal end 321 of the hemostasis valve hub 320. Specifically, the hemostasis valve hub 320 comprises a first arm 326 engaged with a primary cap 360 positioned adjacent a primary seal 352 which are both engaged with a lock nut 344, and the lock nut 344 capable of engaging with a seal 350 positioned within a first lumen 332 of the hemostasis valve hub 320 to axially secure a medical device, such as a catheter. Similar to the seal 250 described with reference to FIG. 4, the seal 350 may have a lumen 351 with a diameter that decreases after axial compression forces act against the seal 350. The lock nut 344 includes an inner portion 346 and an outer portion 348 and the inner portion 346 is configured for engagement with the seal 350. The hemostasis valve hub 320 additionally comprises a second arm 328 defining a second lumen 334 having a secondary seal 354 engaged with the second arm 328 and a secondary cap 362. However, the hemostasis valve hub 320 differs from the hemostasis valve hub 220 in that a distal end 321 of the hemostasis valve hub 320 comprises a narrowed portion 370 that is threaded and capable of engaging with a sheath shaft securing mechanism, illustratively, a threaded cap 372. The threaded cap 372 works to engage with the proximal end 106 (FIG. 1). In particular, the threaded cap 372 is used to connect introducer sheath 100 with hemostasis valve hub 320. However, various other appropriate securing mechanisms may be used for securing the introducer sheath 100 within the hemostasis valve hub 320. With reference to FIGS. 7-10, various of the above referenced elements will be described in further detail.

Specifically, FIG. 7 illustrates an enlarged view of the primary cap 260 described above with reference to the hemostasis valve hub 220. The primary cap 260 comprises a first portion 264 and a second portion 266. The second portion 266 comprises an outer cylinder 267 and a plurality of inner engaging members 269 configured for engaging with external devices, for example tightening ports, tightening devices, sealing devices, and/or sterile sleeves for positioning on the hemostasis valve hub 220. In some embodiments, the primary cap 260 may be configured to couple to a tightening port (not shown) and used to secure a medical device, such as a catheter, passing through the hub 220, for example to prevent axial movement of the catheter. The primary cap 260 may be formed of polycarbonate or polypropylene. The primary cap 260 may be formed of various other materials and the above materials are provided for example. While described with reference to primary cap 260, the above features may apply to the primary cap 360 illustrated with reference to hemostasis valve hub 320.

FIG. 8 illustrates an enlarged view of the lock nut 244. As previously described, the lock nut 244 comprises an inner portion 246 and an outer portion 248 for threadedly engaging with the first arm 226 (FIG. 4) of the hemostasis valve hub 220 (FIG. 4), wherein the outer portion 248 may have a ribbed configuration to enable grasping of the lock nut 244 by the operator to tighten and couple the lock nut 244 onto the first arm 226. The lock nut 244 may further include a threaded surface on an inner surface 249 of the outer portion 248 to threadedly engage with the first arm 226. Lock nut 244 may also include an engagement surface (not shown) on an inner or outer surface of the outer portion 248 to engage in a secured connection with the primary cap 260. For example, a friction fit type of connection may be used to connect lock nut 244 and primary cap 260. Additionally, the inner portion 246 may be configured for engaging with the seal 250 (FIG. 9) upon insertion of the lock nut 244 into the first lumen 232 (FIG. 4) of the first arm 226. As the operator inserts the lock nut 244 into the first arm 226 and couples the lock nut 244 and the first arm 226, the inner portion 246 pushes against the seal 250 and axially pushes the seal 250 through the base 221 until the seal 250 contacts the portion of the base 221 that transitions from the second diameter D2 to the first diameter D1. Once the seal 250 is positioned against the transition portion between the second diameter D2 and the first diameter D1, continued axial pushing of the seal 250 with the inner portion 246 causes axial compression of the seal 250. In turn, and as described herein, axial compression of the seal 250 causes a reduction in the size of the lumen 251 of the seal 250.

FIG. 9 illustrates an enlarged view of the seal 250. As illustrated, the seal 250 has the initial diameter D4 and an initial thickness T1 and the lumen 251 has an initial diameter D3′. After being axially pushed into the smaller lumen section of the first lumen, and with continued axial compression of the seal 250, the initial diameter D3′ of lumen 251 will decrease to the compressed diameter D3 shown in FIG. 4. This ability of the lumen 251 of seal 250 to decrease radially inwardly allows for axial securement of the medical device passing through it, for example the catheter, within the first lumen 232. In other words, as the operator compresses the seal 250 with axial movement of the lock nut 244, the corresponding decrease in the size of the lumen 251 causes or furthers the contact and/or sealing between the seal 250 and the catheter. In addition, the seal 250 may be pressed outwardly against an inner surface of the first arm 226. Thus, the seal 250 may additionally contribute to providing hemostatic sealing of the introducer sheath 100 and the hemostasis valve hub 220. The primary seal 252 (FIG. 4) and the secondary seal 254 (FIG. 4) additionally contribute to the capability for providing the hemostatic seal of the introducer sheath 100 and the hemostasis valve hub 220. The primary seal 252 and secondary seal 254 will be described in further detail herein with reference to FIGS. 10A and 10B.

With reference to FIGS. 10A and 10D, the primary seal 252 and the secondary seal 254 are illustrated and described in further detail. For example, as illustrated, the primary seal 252 may have a diameter D5 that is larger than a diameter D6 of the secondary seal 254. For example, the diameter D5 of the primary seal 252 may have a value of between approximately 9 mm and approximately 11 mm, while the diameter D6 of the secondary seal 254 may have a value of between approximately 5 mm to 8.5 mm. Additionally, the primary seal 252 may have a thickness T2 (FIG. 6) that is larger than a thickness T3 (FIG. 6) of the secondary seal 254. For example, the thickness T2 of the primary seal 252 may have a value ranging between approximately 1.5 mm and approximately 2.5 mm. The thickness T3 of the of the secondary seal 254 may range between approximately 1.25 mm to approximately 2.0 mm. The primary and secondary seals 252, 254 may each be a cylindrical seal such that the primary and secondary seals 252, 254 have a circular cross-section. Further, each of the primary seal 252 and the secondary seal 254 has a partial cross slit within the center of the primary and the secondary seals 252, 254. With reference specifically to the primary seal 252 and to FIGS. 10A-10C, the primary seal 252 may have partial cross slits having a length L2 of approximately 4.5 mm. With reference to the secondary seal 254 shown in FIG. 10D, the secondary seal 254 may have partial cross slits having a length L3 of approximately 1.5 mm. However, the lengths L2, L3 may be varied. For example, the length L2 may range from approximately 3.0 mm to 4.5 mm. The length L3 may range from approximately 1.5 mm to approximately 3.0 mm. FIG. 10B illustrates a cross sectional view of the primary seal 252 of FIG. 10A taken along line C-C and FIG. 10C illustrates a cross sectional view of the primary seal 252 taken along line B-B. As illustrated, the partial cross slit forms when seal is slit cut at 90 degree orientation on opposite faces with a defined slit cut depth d and an overlap O. For example, the partial cross slit of the primary seal 252 may have an overlap O1 with a value of approximately 0.4 mm and slit depth d1 approximately 0.9 mm. FIG. 10E illustrates a cross sectional view of the secondary seal 254 of FIG. 10D taken along line C-C and FIG. 10C illustrates a cross sectional view of the primary seal 252 taken along line B-B. As illustrated, the partial cross slits of the secondary seal 254 may have an overlap O2 with a value of approximately 0.25 mm and slit depth d2 approximately 0.8 mm. However, the overlaps O1, O2 and the depths d1, d2 may be varied. For example, overlap O1 may range from 0.3 mm to 0.5 mm, the depth d1 may range from 0.7 mm to 1.1 mm, the overlap O2 may range from 0.15 mm to 0.35 mm, and the depth d2 may range from 0.6 mm to 1.0 mm. However, the primary and the secondary seals 252, 254 may have various other configurations. For example, the primary and the secondary seals 252, 254 may have an oval cross-sectional shape. The primary seal 252 and the secondary seal 254 may each be composed of silicone, or various other suitable materials like polymer, thermoset, rubber, thermoset elastomer (TSE), or silicone rubber.

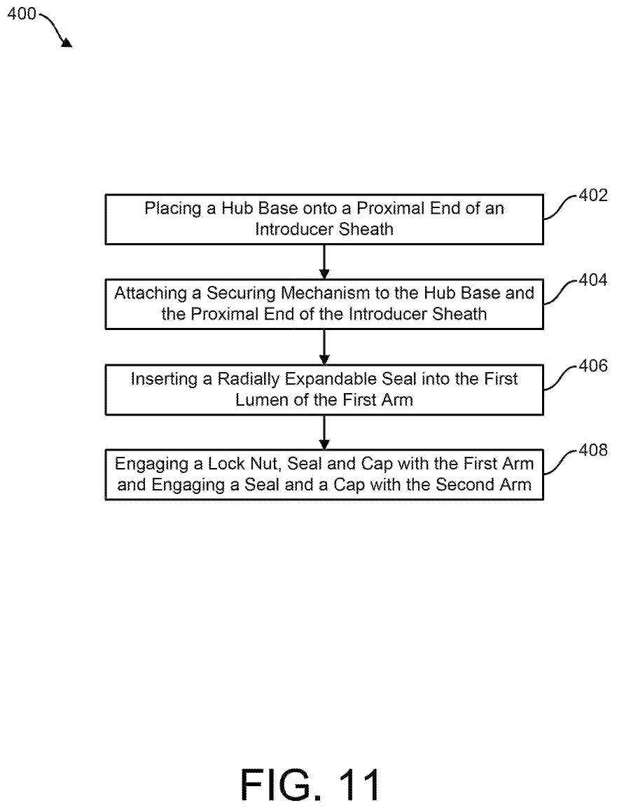

With reference to FIG. 11, a method 400 of assembling an introducer sheath and a hub, for example the introducer sheath 100 and the hub 220 will be described. At block 402, the method 400 first includes placing the hub base 221 onto the proximal end 106 of the introducer sheath 100. More specifically, the introducer sheath 100 is placed into the hub base 221 until the proximal end 106 of the introducer sheath 100 is located at the distal end 222 of the base 221. At block 404, the method 400 further includes attaching a securing mechanism to the hub base 221 and the proximal end 106 of the introducer sheath 100. In various embodiments, the securing mechanism may be the ferrule 231 positioned within the hub base 221 at the distal end 222 of the hemostasis valve hub 220. In this way, after the insertion of the introducer sheath 100 into the base 221, the ferrule 231 may be inserted into the base 221 to hold the introducer sheath 100 in place. In other embodiments, the distal end 222 of the hemostasis valve hub 220 may comprise the narrowed portion 370 and the securing mechanism comprises the threaded cap 372 configured for engagement with the narrowed portion 370.

At block 406, the method 400 further includes inserting the seal 250 into the first lumen 232 of the first arm 226.

At block 408, the method then includes engaging the lock nut 244, the primary seal 252 and the primary cap 260 with the first arm 226 of the hemostasis valve hub 220. This step may include placing the lock nut 244 at least partially within the hub base 221 without completely securing the lock nut 244. This step additionally includes engaging the secondary seal 254 and the secondary cap 262 with the second arm 228 of the hemostasis valve hub 220. As previously described, the configuration of the primary seal 252 and the secondary seal 254 contribute to the hemostatic sealing between the introducer sheath 100 and the hemostasis valve hub 220.

In various embodiments, the introducer sheath 100 and the hub 220 may be used by a physician or operator for inserting at least one medical device into a blood vessel. For example, the introducer sheath 100 and the hub 220 may be used for inserting at least one catheter into the blood vessel V. For example, in use, a method of inserting the at least one medical device may include first inserting the introducer sheath 100 into the blood vessel V. The method may then include inserting a medical device into the first arm 226 of the hemostasis valve hub 220. In various embodiments, the medical device is the blood pump 150 (FIG. 2) and the catheter 170 (FIG. 2) attached to the blood pump 150. Inserting the blood pump 150 and catheter 170 may include inserting the blood pump 150 and the catheter 170 into the first lumen 232 and through the primary seal 252 such that the partial cross slits surround and seal the catheter 170 after the blood pump 150 has been pushed distally through the hub 220 and into the introducer sheath 100. To further seal and maintain the axial positioning of the catheter within the hemostasis valve hub 220 during use, the method may further include securing the axial positioning of the medical device by adjusting the lock nut 244 of the first arm 226. For example, tightening the lock nut 244 causes axial compression of the seal 250, which can thus cause expansion of the seal 250 to radially compress against the medical device, i.e., the catheter, and to radially and/or axially compress against the inner surface of the first arm 226. In various embodiments, a second smaller medical device may be inserted into the second arm 228 of the hemostasis valve hub 220. For example, the second medical device may be a guide catheter for receiving guide tools and/or any other applicable tools that may be desired for use with the hemostasis valve hub 220. In this way, even if there is a first medical device inserted though the first arm 226 of the hub 220, a second medical device can be inserted into the second arm 228 simultaneously. Specifically, once the second medical device is inserted through the second arm 228, the second medical device passes through the secondary seal 254 to maintain a liquid seal between the second medical device and the hub 220. The second medical device can then be extended though the hub 220 and into the introducer sheath 100. In other embodiments, a tightening port (not shown) may be coupled to primary cap 260 to facilitate securing a medical device with respect to hub 220, for example by preventing axially movement of the device with respect to the hub 220.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above-described features.

1. A hemostasis valve hub for a sheath, comprising:
a hub base including a first arm defining a first lumen and having a distal end and a proximal end and a second arm extending from the first arm and defining a second lumen and having a distal end and a proximal end;
a seal positioned within the first lumen and adjacent a lock nut, the lock nut engaged with the proximal end of the first arm and positioned at least partially within the first lumen;
a primary seal positioned adjacent the proximal end of the first arm and a primary cap engaged with the lock nut; and
a secondary seal positioned adjacent the proximal end of the second arm.

2. The hemostasis valve hub of claim 1, wherein the hemostasis valve hub further comprises a securing mechanism engaged with the hub base to secure the hemostasis valve hub to the sheath.

3. The hemostasis valve hub of claim 2, wherein the securing mechanism is a ferrule positioned within the hub base.

4. The hemostasis valve hub of claim 2, wherein the hub base comprises a narrowed portion and the securing mechanism includes a threaded cap engaged with the narrowed portion and the sheath.

5. The hemostasis valve hub of claim 1, wherein the lock nut comprises an inner portion configured to engage with the seal.

6. The hemostasis valve hub of claim 5, wherein the inner portion of the lock nut is configured to axially compress the seal to decrease the size of a lumen of the seal.

7. The hemostasis valve hub of claim 1, wherein the lock nut is configured to threadedly engage with the first arm.

8. The hemostasis valve hub of claim 1, wherein the primary seal has a diameter and the secondary seal has a diameter, the diameter of the primary seal being greater than the diameter of the secondary seal.

9. The hemostasis valve hub of claim 1, wherein the primary seal has a thickness and the secondary seal has a thickness, the thickness of the primary seal being greater than the thickness of the secondary seal.

10. A delivery system for a plurality of medical devices into a blood vessel, comprising:
a sheath for insertion into the blood vessel, the sheath having a proximal end and a distal end; and
a hemostasis valve hub for attachment to the proximal end of the sheath, the hemostasis valve hub including:
a hub base including a first arm defining a first lumen and having a distal end and a proximal end and a second arm extending from the first arm and defining a second lumen and having a distal end and a proximal end;
a seal positioned within the first lumen and adjacent a lock nut, the lock nut engaged with the proximal end of the first arm and positioned at least partially within the first lumen;
a primary seal positioned adjacent the proximal end of the first arm and a primary cap engaged with the lock nut; and
a secondary seal positioned adjacent the proximal end of the second arm.

11. The delivery system of claim 10, wherein the primary seal has a first diameter, the secondary seal has a second diameter, and the first diameter is greater than the second diameter.

12. The delivery system of claim 10, wherein the hub base comprises a securing mechanism for securing to the proximal end of the sheath.

13. The delivery system of claim 12, wherein the securing mechanism is a ferrule positioned within the hub based of the hemostasis valve hub.

14. The delivery system of claim 12, wherein the securing mechanism is composed of a narrowed portion of the hub base having a threaded portion and a threaded cap configured for attachment to the threaded portion.

15. The delivery system of claim 10, wherein the engagement of the lock nut and the seal causes a lumen of the seal to decrease in size.

16. A method of assembly for a sheath and a hub, comprising:
placing a hub base onto a proximal end of a sheath, the hub base having a first arm and a second arm, the first arm defining a first lumen and a second arm defining a second lumen;
attaching a securing mechanism to the hub base and the proximal end of the sheath;
inserting a seal into the first lumen of the first arm; and
engaging a lock nut, a primary seal and a primary cap with the first arm and engaging a secondary seal with the second arm.

17. The method of claim 16, wherein the seal includes a lumen configured to receive a medical device.

18. The method of claim 16, wherein the securing mechanism is a ferrule positioned within the hub base.

19. The method of claim 16, wherein the securing mechanism includes a threaded cap configured for engaging with the proximal end of the sheath and a narrowed portion of the hub base.

20. The method of claim 16, wherein the lock nut comprises an inner portion configured to engage with the seal and axially compress the seal to decrease the size of the lumen of the seal.
